(19) United States
(12) Patent Application Publication
(10) Pub. No.: US 2006/0106451 A1
(43) Pub. Date: May 18, 2006

(54) ELECTRONIC ANTI-COAGULATION STENT FOR INTRA-ARTERIAL DEPLOYMENT

(76) Inventor: Yuri Busiashvili, Pacific Palisades, CA (US)

Correspondence Address: RALPH D CHABOT, 2310 E PONDEROSA DR, SUITE 4, CAMARILLO, CA, 93010, US

(21) Appl. No.: 10/904,615

(22) Filed: Nov. 18, 2004

(51) Int. Cl. A61F 2/90; A61N 1/05

(52) U.S. Cl. 623/1.15; 607/50; 623/1.43; 623/1.44

(57) ABSTRACT

An electronic anti-coagulation stent structure is disclosed. The stent structure comprises a pair of coaxial metal stents having a layer of dielectric material between the stents. A battery is operatively connected preferably near or adjacent to the upstream end upon deployment of the stent. The positive battery terminal establishes an electrical connection to the outer metal stent and the negative terminal establishes an electrical connection to the inner metal stent and this exhibits a capacitor-like properties. The inner metal stent, being negatively charged, promotes a platelet repellent, anti-thrombotic effect.

### TECHNICAL FIELD

The invention pertains to the stent specifically designed for prevention of arterial re-stenosis or re-thrombosis, following arterial stenting.

### BACKGROUND OF THE INVENTION

The anatomical substrate of arterial stenosis is usually either a dissected or injured arterial inner lining, or intima. The damaged intima condition can be affected by natural pathological development such as atherosclerotic plaque, or by invasive intervention, such as balloon angioplasty or stenting.

Critical narrowing of an arterial pathway or its total occlusion can lead to life threatening complications, such as heart attack, arrhythmia, or death.

To prevent such complications, coronary stenting is used to treat critical coronary disease. Re-stenosis occurs in 30-40% of all coronary stenting procedures. Re-stenosis can be successfully reduced in many procedures by the use of drug eluting stents. But even drug eluting stents do not prevent the development of new stenosis caused by the invasive intervention, such as stent edge stenosis, or clot re-growth at the site of an acute total coronary occlusion. Drug eluting stents do not prevent thrombosis.

A deployed stent should optimally achieve two therapeutic goals in the treatment of arterial occlusive disease: 1) immediate relief of arterial stenosis; and, 2) prevention of arterial narrowing and clot formation at the same arterial site. In rare circumstances, a solid-sleeve stent, rather than a fenestrated stent, is required to seal an accidental puncture in the arterial wall caused by instrumentation. An example of a solid-sleeve stent is the JOSTENT®, manufactured by JOMED Inc., Rancho Cordova, Calif.

In many situations, a blood clot propagates along an extended portion of an artery. Accordingly, since stents can not be deployed along such extended portions, a stent is typically deployed across the target lesion.

It has been reported that all cells and surfaces of the body possess an electrical charge. Healthy arterial intima is negatively charged against platelets. Because of this, healthy intima is not thrombogenic. Trauma to the arterial wall by pathological process or instrumentation will cause the injured area to become positively charged with a concomitant thrombosis at the site of the injury.

Intima disrupted either by natural pathophysiologic processes, such as acute myocardial infarction, or by therapeutic intervention, such as percutaneous transluminal coronary angioplasty (PTCA) or stenting, is thrombogenic as it is positively charged against platelets and attracts them, instigating clot formation.

Besides trauma, even a simple incision into a blood vessel will create a positive charge at the site of the incision. However, if the incision can be maintained negatively charged by the application of an electrical current, coagulation at the site will be inhibited and the wound will continue to ooze for many hours. If an applied current is positive rather than negative, clotting will accelerate. Electronic Anti-hemocoagulation, Biomat. Med. Dev. Art. Org. (1986); 14(3&4) pp. 195-225, Delangis P A, Yen T.

Thrombogenicity of the surface, contacting with blood, depends on several factors, among which of significance is the electric charge. A positive charge facilitates thrombus formation and a negative one hinders formation. Apart from the charge value and sign, surface homogeneity of the charge, as well as its stability, were shown to play a role in these processes. An Assessment of Athrombogenic Properties of Electret Polyethylene Film, Polymers in Medicine (1998) T. XXVIII, Nr 1-2, pp. 3-13, Bozena Lowkis, Maria Szymonowicz.

Recently, stents have been developed which incorporate a polymer coating or sheeting about the expandable stent which can stretch as the stent expands. More importantly, these polymer coatings are negatively charged, thus having an anti-thrombotic property. References describing the negatively charged characteristic of polymer coatings include U.S. Pat. No. 6,228,845 and U.S. Pat. No. 5,833,651 both issued to Donovan et al.

Microturbulance at the site of the stented arterial intima is an additional hemodynamic factor, contributing to the re-stenosis or re-thrombosis condition.

Currently available drug eluting stents fail to address the issue of re-thrombosis in acute coronary occlusion and do not prevent re-stenosis at the edges of a deployed stent as a result of excessive balloon inflation.

### BRIEF SUMMARY OF THE INVENTION

A coaxial stent structure (CSS) is presented. The CSS is comprised of a pair of coaxial stents, one within the other and having insulation material, preferably a layer of insulation material, sufficient to prevent conductivity between the stent pair. A power supply is provided where one terminal is connected to a respective stent so that a positive charge is applied to the stent in direct contact with the arterial wall and a negative charge is applied to the stent in direct contact with arterial blood flow.

In order to prevent re-growth of the clot, either spontaneous or iatrogenic, the CSS should be capable of maintaining a negative charge over an extended period of time. The reason for maintaining the negative charge is to prevent regrowth of a clot in the target artery.

Although application of current to a stent has been tried prior to deployment, current has never been delivered to a deployed stent. Application of the current is complicated by the fact that blood conducts electric current and any power source such as a battery operably connected to a deployed stent would lose its charge within an unacceptably short time if the sole purpose of the battery was to generate an electric current through a deployed stent.

In order for the CSS to retain a negative charge over a period of time, the CSS must be designed to function like a capacitor. In other words, each coaxial stent must be designed to represent one of the two plates of the capacitor, where each coaxial stent is isolated from the other by any dielectric material that does not conduct electric current.

The dielectric insulating material can be made of any number of polymeric materials. Alternatively, the insulating material could also be an electret.

The insulating material is “sandwiched” or positioned between the two concentric metal stents, one inside another, like coaxial cylinders. The electret or other dielectric material functions the same as the dielectric material found in a capacitor. According to my invention, the coaxial stents will play the role of capacitor plates. Construction of the CSS should be simple, allowing easy trackability.

The insulating material must be designed to prevent conductivity between the pair of coaxial stents. A dielectric layer is disposed between the coaxial stent pair. Because deployment of the CSS will radially extend outward the coaxial stent pair, the insulating material must be able to stretch without degradation of its insulating properties.

In practice, the CSS would be deployed in the target artery together with a power source such as a mini-battery that is preferably located on the proximal end. Deployment of the CSS is accomplished by procedure well known to those having skill in the art. The positive and negative battery terminals are connected to respective coaxial stents upon their expansion in the target artery. Since the negative terminal is connected to the inner stent, a negative electrostatic charge is sustained creating a condition called electronic anticoagulation.

Electronic anticoagulation is achieved if the inner surface of the inner deployed coaxial stent has a negative static electric charge, as long as it is greater than the charge carried by the platelets. The platelets then will not be attracted to this portion of the deployed stent; clot formation will be prevented.

As described earlier, my coaxial stent structure comprises a pair of stents; one stent having a slightly smaller target diameter that is positioned inside the second stent, thereby forming parallel cylindrical coaxial plates. Between the stents, an insulating dielectric material or electret occupies this annular region.

The negative electrostatic charge imposed on the inner coaxial stent and the positive electrostatic charge imposed on the outer coaxial stent is achieved by connection to a power supply such as a battery, preferably permanently connected and located preferably near or adjacent the upstream end of the stent. When the positive and negative terminals of the battery contact the respective plates (i.e. respective stents), voltage is applied to both the inner and outer coaxial stents and each become polarized.

A static electrical charge is maintained to both the inner and outer coaxial stents.

The battery could be mounted on the same balloon, that delivers the coaxial stents, or the battery can be positioned on a separate balloon and thereafter operably connected to the deployed inner and outer stents.

Before the coaxial stents are actually deployed and connected to the battery, there is no current flow through the circuit. The coaxial stents are designed so that current can only flow upon deflation of the delivery balloon and deployment of the stent. As each stent is expanded, the contact points between each stent and a respective battery terminal are established. In this manner, the electrical circuit is completed and current flows. A negative charge soon builds up on the inner coaxial stent and a positive charge builds up on the outer coaxial stent. The dielectric material sandwiched between each coaxial stent prevents discharge across each stent.

As the voltage across each coaxial stent increase, the driving voltage of the battery is reduced by the amount that the capacitor voltage has increased. Eventually, the voltage across the coaxial stents approaches the battery voltage and the circuit current approaches zero. In this manner, the coaxial stents are charged, the outer coaxial stent retaining the positive electrostatic charge, while the inner coaxial stent retains a negative electrostatic charge.

In order to deploy the stent primarily, without predilatation with the balloon, the stent should be easily delivered to the target lesion and across the stenosis. Even though contemporary stents are low profile stents with excellent deliverability and scaffolding, it is not always possible. Since my coaxial stent design is more complex than a single stent, it is quite possible my design may lose some maneuverability. In order to avoid such a disadvantage, certain construction criteria is recommended. Both inner and outer coaxial stents are preferably constructed with a plurality of connected sinusoidal rings; a stent design which is well known in the prior art.

Preferably, the inner and outer stent designs will permit the inner stent to have substantially the same inner diameter as the outer stent design upon deployment. Each stent is comprised of expandable rings that are spaced apart from one another and are connected by intermittent linking stems. The spacing of the rings comprising one stent are sufficient to allow the interconnected rings of the other stent construction cylinder to fill the spacing and thereby permit adequate maneuverability of the CSS.

Together with the dielectric material layer located between coaxial stents, the construction of CSS will be completed, when attached to the battery.

### BRIEF DESCRIPTION OF THE DRAWINGS

FIG. 1 is a schematic view of my novel coaxial stent structure.

FIG. 2 is a view taken along line 2-2 of FIG. 1.

### DETAILED DESCRIPTION OF THE INVENTION

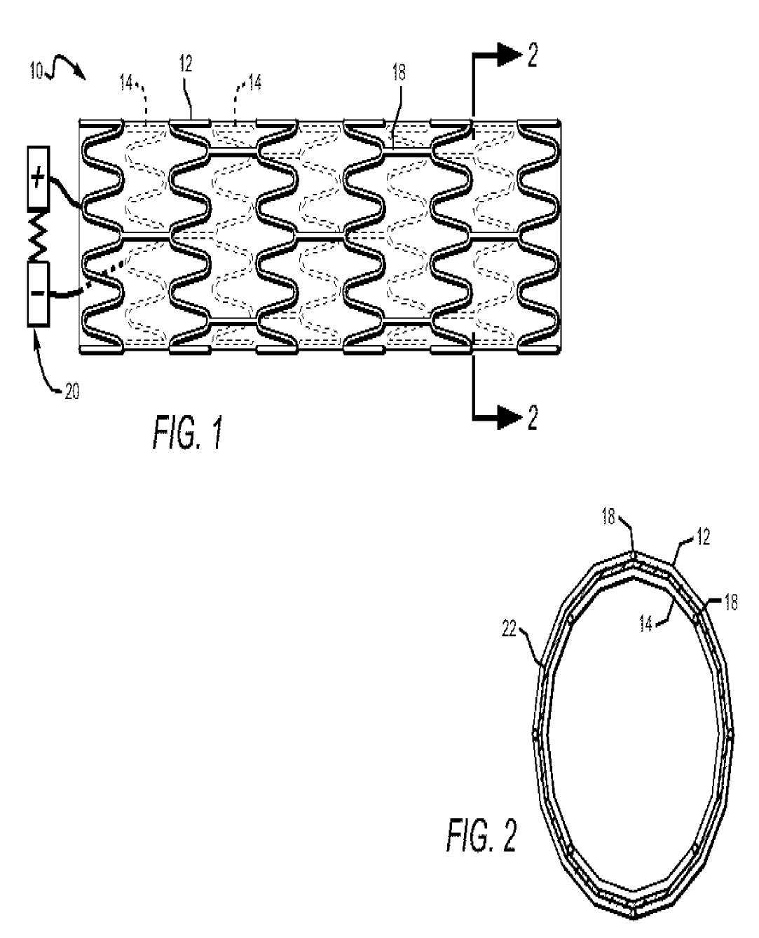

FIG. 1 provides a schematic side view representation of my coaxial stent structure 10 which comprises an outer stent 12 and an inner stent 14. Stents 12 and 14 are each comprised of a plurality of rings which are sinusoidal in configuration and thus capable of outward expansion. The rings of each stent are connected to adjacent rings by stems 18.

When the stent structure is deployed, battery 20 becomes operatively connected, its positive terminal connected to outer stent 12 and its negative terminal connected to inner stent 14 upon deployment. It is to be understood that the position of battery 20 illustrated in FIG. 1 is shown to simplify explanation. In the preferred embodiment, battery 20 is preferably physically attached as part of the stent structure near or adjacent the upstream end.

As best seen in the cross-sectional view of FIG. 2, coaxial stent structure 10 further comprises an insulation layer 22 to prevent conductivity between stents 12 and 14. Insulation layer 22 is made of dielectric material.

Before deployment, sometimes referred to as radial expansion, coaxial stent structure 10 is delivered adjacent to the target lesion by the use of a guide wire as is well known in the art. Battery 20, prior to deployment, is not operatively connected to either stent. The coaxial stents are designed so that upon radial expansion by a balloon, each stent will contact with respective leads of battery 20; thereby resulting in an operative connection between the negative terminal and inner stent 14 and the positive terminal and outer stent 12.

1. A coaxial stent structure comprising:
a pair of coaxial stents;
a battery having its negative terminal operably connected to the inner stent and the positive terminal operably connected to the outer stent; and,
insulation material positioned between said pair of coaxial stents sufficient to prevent conductivity.

2. The coaxial stent structure of claim 1 wherein each of said coaxial stents comprise a plurality of connected sinusoidal rings.

3. The coaxial stent structure of claim 1 wherein said insulation material is a layer of dielectric material.

4. The coaxial stent structure of claim 1 wherein said insulation material is an electret.

5. A coaxial stent structure for intra-arterial deployment in an arterial pathway to relieve arterial stenosis comprising:
a pair of coaxial stents, each stent comprising a plurality of sinusoidal rings connected by a plurality of stems;
a battery having its negative terminal operably connected to the inner stent and the positive terminal operably connected to the outer stent upon intra-arterial deployment; and,
a layer of insulation material positioned between said pair of coaxial stents sufficient to prevent conductivity between said stents.

6. The coaxial stent structure of claim 5 wherein said insulation material is a layer of dielectric material.

7. The coaxial stent structure of claim 5 wherein said insulation material is an electret.

8. A coaxial stent structure for intra-arterial deployment comprising:
a pair of stents in coaxial relationship to one another and suitably sized for intra-arterial deployment;
a battery physically attached to at least one of said stents;
insulation material positioned between said pair of stents sufficient to prevent conductivity; and,
where upon positioning said coaxial stent structure within an artery and said pair of stents are deployed, the negative terminal of said battery becomes operably connected to the inner stent and the positive terminal of said battery terminal becomes operably connected to the outer stent.

9. The coaxial stent structure of claim 8 wherein said insulation material is a layer of dielectric material.

10. The coaxial stent structure of claim 8 wherein said insulation material is an electret.

11. The coaxial stent structure of claim 8 where said battery is attached to one of said stents near or adjacent to the upstream end of said stent.
